(19) United States
(12) Patent Application Publication
(10) Pub. No.: US 2026/0207948 A1
(43) Pub. Date: Jul. 23, 2026

(54) LOCATION VALIDATION

(71) Applicant: Alpha Omega Engineering Ltd., Nof HaGalil (IL)

(72) Inventors: Hagai BERGMAN, Jerusalem (IL); Eliran HADAD, Yokneam Illit (IL); Halen ERDMAN, Givataim (IL); Aviv MIZRAHI-KLIGER, San Francisco, CA (US)

(73) Assignee: Alpha Omega Engineering Ltd., Nof HaGalil (IL)

(21) Appl. No.: 19/138,129

(22) PCT Filed: Dec. 5, 2023
(86) PCT No.: PCT/IL2023/051247

§ 371 Date: Jun. 12, 2025

Related U.S. Application Data
(60) Provisional application No. 63/431,762, filed on Dec. 12, 2022.

(51) Int. Cl. A61N 1/372; A61N 1/05

(52) U.S. Cl. CPC A61N 1/37247; A61N 1/0529

(57) ABSTRACT

The invention relates to a method of providing information to a surgeon about a location of a contact of a lead in a brain of a patient, comprising: receiving a first reading using a microelectrode at a first location; receiving a chosen target location for a lead comprising a contact according to the first reading; receiving at least one second reading at a second location at or near to the received chosen target location using the contact of the lead; performing a correlation between the first reading and the second reading; estimating a distance between the first location and the second location using a result of the correlation; providing information to the surgeon regarding one or both of a distance and a direction between a location of the contact of the lead to the chosen target location.

### RELATED APPLICATION/S

This application claims the benefit of priority of U.S. Provisional Patent Application No. 63/431,762 filed on Dec. 12, 2022, the contents of which are incorporated herein by reference in their entirety.

### FIELD AND BACKGROUND OF THE INVENTION

The present invention, in some embodiments thereof, relates to system and methods for providing information regarding a position of a lead relative to a previously positioned microelectrode and, more particularly, but not exclusively, to a system and methods for providing information regarding a position of a lead relative to a previously positioned and then extracted microelectrode in the brain of a patient.

### SUMMARY OF THE INVENTION

Following is a non-exclusive list including some examples of embodiments of the invention. The invention also includes embodiments which include fewer than all the features in an example and embodiments using features from multipel examples, also if not expressly listed below.

### EXAMPLE 1

A method of providing information to a surgeon about a location of at least one contact of at least one lead in a brain of a patient, comprising:
a. receiving at least one first reading using at least one microelectrode at a first location;
b. receiving at least one chosen target location for at least one lead comprising at least one contact according to said at least one first reading;
c. receiving at least one second reading at a second location at or near to said received chosen target location using said at least one contact of said at least one lead;
d. performing a correlation between said at least one first reading and said at least one second reading;
e. estimating a distance between said first location and said second location using a result of said correlation;
f. providing information to said surgeon regarding one or both of a distance and a direction between a location of said at least one contact of said at least one lead to said chosen target location.

### EXAMPLE 2

The method according to example 1, wherein said providing information to said surgeon comprises providing information to said surgeon regarding a direction to move said at least one contact of said at least one lead to reach said chosen target location.

### EXAMPLE 3

The method according to example 1 or example 2, wherein said providing information to said surgeon comprises providing information to said surgeon regarding a distance to move said at least one contact of said at least one lead to reach said chosen target location.

### EXAMPLE 4

The method according to any one of examples 1-3, wherein said first location is said target location.

### EXAMPLE 5

The method according to any one of examples 1-4, wherein when said at least one lead comprises more than one contact, and said steps c-f are performed for each contact.

### EXAMPLE 6

The method according to any one of examples 1-5, wherein before said performing at least one first reading, the method further comprises inserting said at least one microelectrode to an estimated location within said brain of said patient.

### EXAMPLE 7

The method according to any one of examples 1-6, wherein before said performing at least one second reading, the method further comprises extracting said at least one microelectrode and inserting said at least one lead comprising said at least one contact using a same path as said at least one microelectrode until reaching a location close to said chosen target location.

### EXAMPLE 8

The method according to any one of examples 1-7, wherein said at least one first reading is performed in at least one frequency.

### EXAMPLE 9

The method according to any one of examples 1-8, wherein said at least one frequency is one or more of Beta, Theta and Gamma.

### EXAMPLE 10

The method according to any one of examples 1-9, wherein said at least one second reading is performed in a same frequency as said at least one first reading.

### EXAMPLE 11

The method according to any one of examples 1-10, wherein said performing at least one first reading further comprises obtaining a plurality of microelectrode Power Spectral Densities (PSDs) of said at least one first reading.

### EXAMPLE 12

The method according to any one of examples 1-11, wherein said performing at least one second reading further comprises obtaining at least one lead PSDs of said of said at least one second reading.

### EXAMPLE 13

The method according to any one of examples 1-12, wherein said correlating comprises calculating a group of microelectrode PSDs from said plurality of microelectrode PSDs that match said at least one lead PSDs.

EXAMPLE 14

The method according to any one of examples 1-13, wherein said correlating comprises, after said calculating a group of microelectrode PSDs, averaging values of said calculated group of said microelectrode PSDs.

### EXAMPLE 15

The method according to any one of examples 1-14, wherein said correlating comprises correlating between said averaged PSDs values and said at least one lead PSDs.

### EXAMPLE 16

The method according to any one of examples 1-15, wherein said correlating comprises calculating a hypothetical depth in which said at least one contact of said at least one lead is located based on a result of said correlation.

### EXAMPLE 17

The method according to any one of examples 1-16, wherein said correlating comprises calculating a distance between said at least one contact of said at least one lead to said chosen location based on a result of said correlation.

### EXAMPLE 18

The method according to any one of examples 1-17, wherein said distance is calculated according to a distance between a location of said at least one contact and a location where a result of said correlation is a highest result of said correlation.

### EXAMPLE 19

The method according to any one of examples 1-18, wherein said performing at least one first reading comprises performing a plurality of readings at fixed times using at least one microelectrode.

### EXAMPLE 20

The method according to any one of examples 1-19, wherein said a plurality of readings are performed every 0.1 seconds.

### EXAMPLE 21

The method according to any one of examples 1-20, wherein said performing at least one first reading comprises performing a plurality of readings at fixed depths using at least one microelectrode.

### EXAMPLE 22

The method according to any one of examples 1-21, wherein said plurality of readings are performed every 0.1 millimeters.

### EXAMPLE 23

The method according to any one of examples 1-22, wherein said plurality of microelectrode Power Spectral Densities (PSDs) are obtained for a plurality of depths to which said at least one microelectrode is inserted.

### EXAMPLE 24

The method according to any one of examples 1-23, wherein said plurality of microelectrode Power Spectral Densities (PSDs) are obtained using a pWelch method.

### EXAMPLE 25

The method according to any one of examples 1-24, wherein said correlation is performed using one or more of Beta peak power, Beta AUC, Theta peak power, Theta AUC, Gamma peak power and Gamma AUC.

### EXAMPLE 26

The method according to any one of examples 1-25, wherein said performing a correlation is performed multiple times at different depths.

### EXAMPLE 27

A method of advancing a lead, comprising:
a. performing at least one first reading in at least one frequency using at least one microelectrode;
b. choosing a target location for at least one lead comprising at least one contact;
c. performing at least one second reading in said at least one frequency close to said chosen target location using said at least one contact of said at least one lead;
d. receiving information regarding one or both of a distance and a direction between a location of said at least one contact of said at least one lead to said chosen target location;
e. moving said lead according to said received information regarding said distance and said direction;
f. performing steps c-e until said received information regarding said distance between said location of said at least one contact of said at least one lead to said chosen target location is equal to zero.

### EXAMPLE 28

A system for implanting a lead in a brain of a patient, comprising:
a. at least one microelectrode;
b. at least one lead comprising at least one contact
c. circuitry comprising instructions for:
   i. receiving at least one first reading using at least one microelectrode at a first location;
ii. receiving at least one chosen target location for at least one lead comprising at least one contact according to said at least one first reading;
iii. receiving at least one second reading at a second location at or near to said received chosen target location using said at least one contact of said at least one lead;
iv. performing a correlation between said at least one first reading and said at least one second reading;
v. estimating a distance between said first location and said second location using a result of said correlation;
vi. providing information regarding one or both of a distance and a direction between a location of said at least one contact of said at least one lead to said chosen target location.

### EXAMPLE 29

The system according to example 28, wherein said providing information comprises providing information regarding a direction to move said at least one contact of said at least one lead to reach said chosen target location.

### EXAMPLE 30

The system according to example 28 or example 29, wherein said providing information comprises providing information regarding a distance to move said at least one contact of said at least one lead to reach said chosen target location.

### EXAMPLE 31

The system according to any one of examples 28-30, wherein said first location is said target location.

### EXAMPLE 32

The system according to any one of examples 28-31, wherein when said at least one lead comprises more than one contact, and said steps c-f are performed for each contact.

### EXAMPLE 33

The system according to any one of examples 28-32, wherein before said performing at least one first reading, the method further comprises inserting said at least one microelectrode to an estimated location within said brain of said patient.

### EXAMPLE 34

The system according to any one of examples 28-33, wherein before said performing at least one second reading, the method further comprises extracting said at least one microelectrode and inserting said at least one lead comprising said at least one contact using a same path as said at least one microelectrode until reaching a location close to said chosen target location.

### EXAMPLE 35

The system according to any one of examples 28-34, wherein said at least one first reading is performed in at least one frequency.

### EXAMPLE 36

The system according to any one of examples 28-35, wherein said at least one frequency is one or more of Beta, Theta and Gamma.

### EXAMPLE 37

The system according to any one of examples 28-36, wherein said at least one second reading is performed in a same frequency as said at least one first reading.

### EXAMPLE 38

The system according to any one of examples 28-37, wherein said performing at least one first reading further comprises obtaining a plurality of microelectrode Power Spectral Densities (PSDs) of said at least one first reading.

### EXAMPLE 39

The system according to any one of examples 28-38, wherein said performing at least one second reading further comprises obtaining at least one lead PSDs of said of said at least one second reading.

### EXAMPLE 40

The system according to any one of examples 28-39, wherein said correlating comprises calculating a group of microelectrode PSDs from said plurality of microelectrode PSDs that match said at least one lead PSDs.

### EXAMPLE 41

The system according to any one of examples 28-40, wherein said correlating comprises, after said calculating a group of microelectrode PSDs, averaging values of said calculated group of said microelectrode PSDs.

### EXAMPLE 42

The system according to any one of examples 28-41, wherein said correlating comprises correlating between said averaged PSDs values and said at least one lead PSDs.

### EXAMPLE 43

The system according to any one of examples 28-42, wherein said correlating comprises calculating a hypothetical depth in which said at least one contact of said at least one lead is located based on a result of said correlation.

### EXAMPLE 44

The system according to any one of examples 28-43, wherein said correlating comprises calculating a distance between said at least one contact of said at least one lead to said chosen location based on a result of said correlation.

### EXAMPLE 45

The system according to any one of examples 28-44, wherein said distance is calculated according to a distance between a location of said at least one contact and a location where a result of said correlation is a highest result of said correlation.

### EXAMPLE 46

The system according to any one of examples 28-45, wherein said performing at least one first reading comprises performing a plurality of readings at fixed times using at least one microelectrode.

### EXAMPLE 47

The system according to any one of examples 28-46, wherein said a plurality of readings are performed every 0.1 seconds.

### EXAMPLE 48

The system according to any one of examples 28-47, wherein said performing at least one first reading comprises performing a plurality of readings at fixed depths using at least one microelectrode.

### EXAMPLE 49

The system according to any one of examples 28-48, wherein said plurality of readings are performed every 0.1 millimeters.

### EXAMPLE 50

The system according to any one of examples 28-49, wherein said plurality of microelectrode Power Spectral Densities (PSDs) are obtained for a plurality of depths to which said at least one microelectrode is inserted.

### EXAMPLE 51

The system according to any one of examples 28-50, wherein said plurality of microelectrode Power Spectral Densities (PSDs) are obtained using a pWelch method.

### EXAMPLE 52

The system according to any one of examples 28-51, wherein said correlation is performed using one or more of Beta peak power, Beta AUC, Theta peak power, Theta AUC, Gamma peak power and Gamma AUC.

### EXAMPLE 53

The system according to any one of examples 28-52, wherein said performing a correlation is performed multiple times at different depths.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of an exemplary general method of providing information to surgeon, according to some embodiments of the invention;

FIG. 2 is a flowchart detailing actions performed in general steps 102 and 104 disclosed in the flowchart in FIG. 1, according to some embodiments of the invention;

FIGS. 3, 4, 5 and 6 are flowcharts detailing actions performed in general steps 106, 108 and 110 disclosed in the flowchart in FIG. 1, according to some embodiments of the invention;

FIGS. 7A-7B are exemplary graphs of MER recording PSD, lead recording LFP (Local Field Potential) and an exemplary correlation graph, according to some embodiments of the invention;

FIG. 8 is an exemplary graph, plotting the correlation between recordings made by the microelectrode and the lead in multiple frequencies, according to some embodiments of the invention;

FIG. 9 is a plurality of exemplary calculated correlations for a plurality of frequencies as generated by the system, according to some embodiments of the invention;

FIG. 10 is a flowchart of an exemplary procedure, according to some embodiments of the invention; and

FIGS. 11A-110 are exemplary graphic user interface screenshots related to the exemplary procedure, according to some embodiments of the invention.

### DESCRIPTION OF SPECIFIC EMBODIMENTS OF THE INVENTION

The present invention, in some embodiments thereof, relates to system and methods for providing information regarding a position of a lead relative to a previously positioned microelectrode and, more particularly, but not exclusively, to a system and methods for providing information regarding a position of a lead relative to a previously positioned and then extracted microelectrode in the brain of a patient.

### Overview

An aspect of some embodiments of the invention relates to providing information to a surgeon regarding the position of a lead being inserted in the brain of a patient in order to assess the correct positioning of the lead. In some embodiments, the position of the lead is in relation to a position of a microelectrode previously inserted in the brain of the patient. In some embodiments, the information of the position is based on a correlation between signals recorded by the microelectrode and signals recorded by the lead. In some embodiments, the signals recorded by the microelectrode are a plurality of signal recorded at different distances from the point of insertion of the microelectrode. In some embodiments, the signals are optionally recorded at specific fixed distances from the point of insertion of the microelectrode and/or the lead, for example, recordings are performed at distances of 1 mm between one and another. Optionally at distances of from about 0.5 mm and 1.5 mm, optionally at distances of from about 0.3 mm to about 2 mm. Optionally at distances of from about 0.1 mm and 3 mm. Optionally at distances higher than 3 mm. In some embodiments, the information provided to the surgeon is to whether further insert the lead or retract the lead. In some embodiments, the correlation is performed using the Power Spectral Density (PSD) of the readings. A potential advantage of using the Power Spectral Density (PSD) is that it allows to get a more reliable correlation due to the change in the frequency domain in the chosen target (for example: STM or GP). In some embodiments, the correlation provides an accurate correlation of the position of about ±0.5 mm or less.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Description of the Exemplary Method

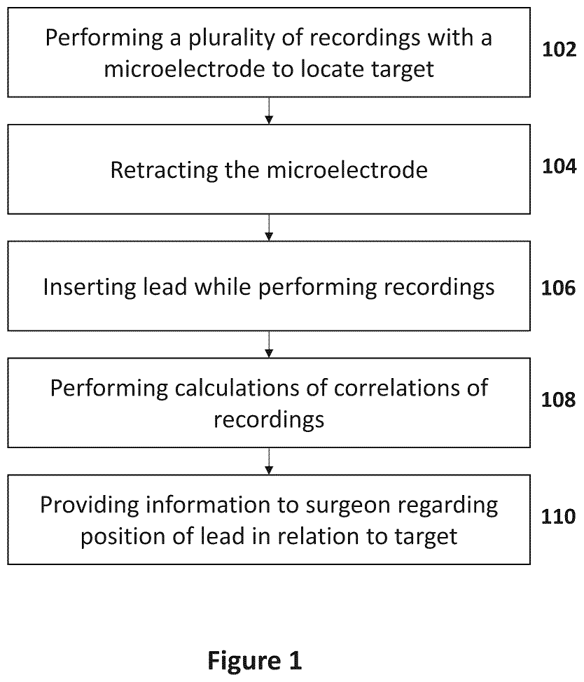

Referring now to the drawings, FIG. 1 shows a flowchart of an exemplary general method of providing information to surgeon, according to some embodiments of the invention.

In the following paragraphs, a general explanation of the exemplary method will be described. After that, a more detail description of the exemplary method will be provided.

In some embodiments, the general method begins by inserting a microelectrode into the patient and performing a plurality of readings during the insertion to assess the location of the target where the lead will be inserted 102.

In some embodiments, once the location of the target is chosen, the microelectrode is retracted 104.

In some embodiments, a lead is then inserted in the same path as the microelectrode, and recordings are made either form the beginning of the insertion or close to the location where the chosen target is located 106.

In some embodiments, the system automatically performs calculations of correlations between the readings recorded with the microelectrode and the reading being recorded with the lead 108.

In some embodiments, based on the results of the calculations of the correlations, the system provides information to the surgeon regarding the current position of the lead in relation to the position of the target as measured by the microelectrode. In some embodiments, the information is supplied as a distance that the lead needs to be moved in order to arrive at the target location 110.

### Exemplary Detail Description of the Exemplary Method

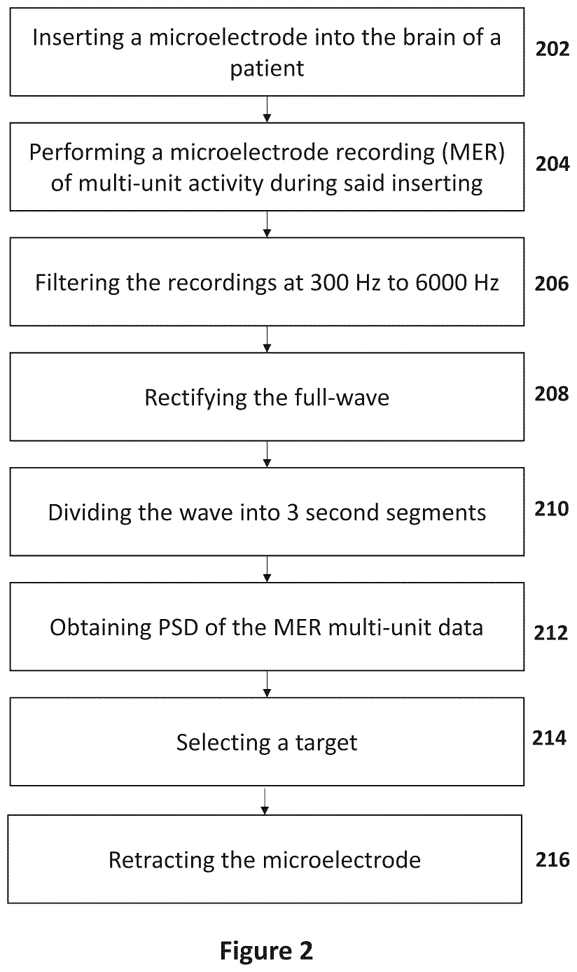

Referring now to FIG. 2, showing a flowchart detailing actions performed in general steps 102 and 104 disclosed in the flowchart in FIG. 1, according to some embodiments of the invention.

In some embodiments, the microelectrode is inserted into the brain of the patient 202. In some embodiments, the insertion of the microelectrode is preceded by a preoperative magnetic resonance imaging to identify fiducial points and anatomic landmarks. For example, the anatomical location of the anterior commissure, posterior commissure and midsagittal plane are determined and the nominal coordinates of the target are obtained.

In some embodiments, the microelectrode recording (MER) of multi-unit activity (MUA) is performed during the insertion of the microelectrode 204. In some embodiments, recordings are performed at fixed times and/or fixed depths. For example, recordings are performed every 1 sec (optionally every time lapse of from about 0.5 sec to about 1.5 sec; optionally from about 0.3 sec to about 2 sec; optionally from about 0.1 sec to about 3 sec) and/or at every 0.1 mm (optionally every distance of from about 0.05 mm to about 0.15 mm; optionally from about 0.03 mm to about 0.2 mm; optionally from about 0.01 mm to about 0.3mm) that the microelectrode is inserted.

In some embodiments, the recordings are filtered at frequencies from about 300 Hz to about 9000 Hz 206.

In some embodiments, filtering the recording is performed to make sure that the signals are MUA and not a Local Field Potential (LFP). In some embodiments, an additional reason to perform the filtering is that it potentially helps avoiding recording electrical noise found in the operating room.

In some embodiments, the full wave is rectified 208.

In some embodiments, the wave is divide in 3 second segments 210. In some embodiments, a potential advantage of dividing in 3 second segments is that using a frequency range of ⅓ Hz allows to look at a narrow range like Beta or Gamma and get a good resolution in this range. In some embodiments, dividing in 3 second segments is performed in order to use the frequency range of ⅓ Hz.

In some embodiments, the Power Spectral Density (PSD) of the MER multi-unit data is obtained 212. In some embodiments, the PSD is obtained for a plurality of depths to which the microelectrode is inserted. In some embodiments, pWelch method is used to calculate the different PSDs using a frequency resolution, for example, of ⅓ Hz. In some embodiments, the different PSDs are normalized by division in total power in a range, for example, of from about 0.1 Hz to about 60 Hz. In some embodiments, the correlation can be done using other data, for example, raw readings data. In some embodiments, a potential advantage of using the Power Spectral Density (PSD) of the MER multi-unit data is that it allows to choose a target with a variant frequency so the frequency domain can be correlated.

In some embodiments, the target is then selected 214.

In some embodiments, the microelectrode is then extracted 216.

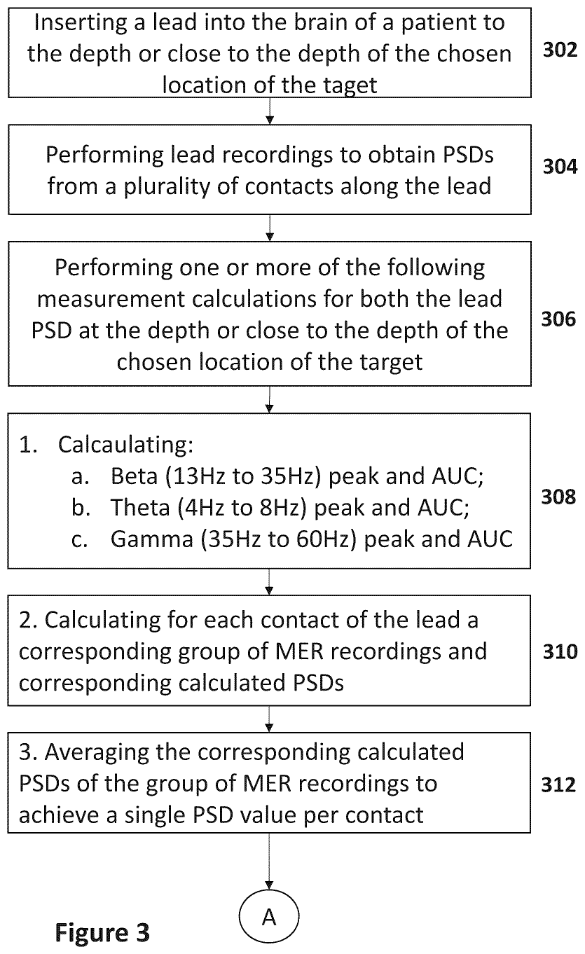

Referring now to FIG. 3, showing a flowchart detailing actions performed in general steps 106, 108 and 110 disclosed in the flowchart in FIG. 1, according to some embodiments of the invention.

In some embodiments, the lead is inserted to or close to the location (depth) where the target was chosen 302.

In some embodiments, led recordings are performed to obtain PSDs from a plurality of contacts along the led 304. In some embodiments, the number of contacts depends on the type of led used. In some embodiments, the number of contacts can be for example 2 contacts, 3 contacts, 4 contacts, 5 contacts, 6 contacts or more. In some embodiments, the PSDs obtained are for that specific location where the lead was inserted, which is close to or at the chosen target (contrary to the recordings performed during the MER recordings that were obtained at multiple depth locations during the insertion of the microelectrode),

In some embodiments, one or more of the following measurement calculations for both the lead PSD at the depth or close to the depth of the chosen location of the target 306:

In some embodiments, one or more of the following is calculated: 1. Beta (13 Hz to 35 Hz) peak power and area under the curve (AUC); 2. Theta (4 Hz to 8 Hz) peak power and area under the curve (AUC); and 3. Gamma (35 Hz to 60 Hz) peak power and area under the curve (AUC) 308. In some embodiments, only calculated peak powers are used in the correlation assessment. In some embodiments, only calculated AUC are used in the correlation assessment. In some embodiments, a mix of peak powers and AUCs are used in the correlation assessment. See below considerations when to use peak power and when to use AUC.

Leads usually comprise more than one contact along the lead. In some embodiments, once the lead is inserted, each contact of the lead is located at a different depth, which corresponds to a distinct group of similar MER recordings at those depths. The reason that the corresponding MER recording are a group of MER recordings (and not a single MER recording) is that usually each contact comprises a width of about 1.5 mm, and the MER recordings are performed, for example, about every 0.1 mm (see above), therefore, a group of MER recordings extending a length of about the same length of the contact are used for each corresponding contact. Therefore, the next action is to calculate a group of MER recordings that match the recordings for each of those contacts in the lead 310.

In some embodiments, after the group of MERs are found for each contact in the lead, the PSD measures of those MERs are averaged together to obtain a single averaged PSD value per contact 312.

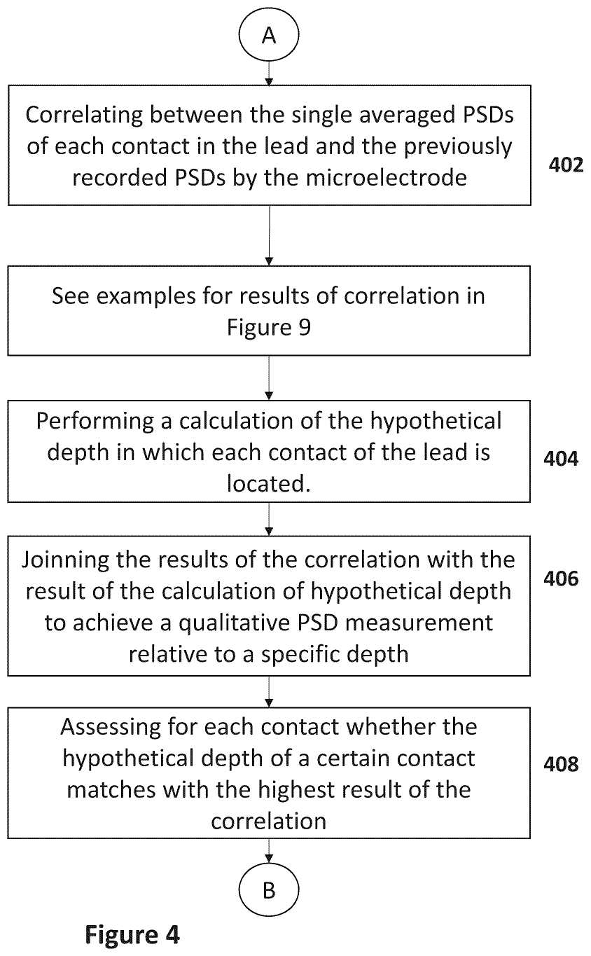

The flow chart continues following the letter “A” to FIG. 4.

In some embodiments, once a single averaged PSD value is found for each contact of the lead, the next action is correlating between those single averaged PSD values and the PSD recordings performed with the microelectrode 402.

See below exemplary detail explanations on the calculations made for the correlation.

In some embodiments, the next action is performing a calculation of the hypothetical depth in which each of the contacts of the lead are located 404.

In some embodiments, the result of the calculated hypothetical depth is joined with the results of the correlation 406.

In some embodiments, the next step is assessing for each contact whether the calculated hypothetical depth of each contact of the lead matches with the highest result of the correlation 408.

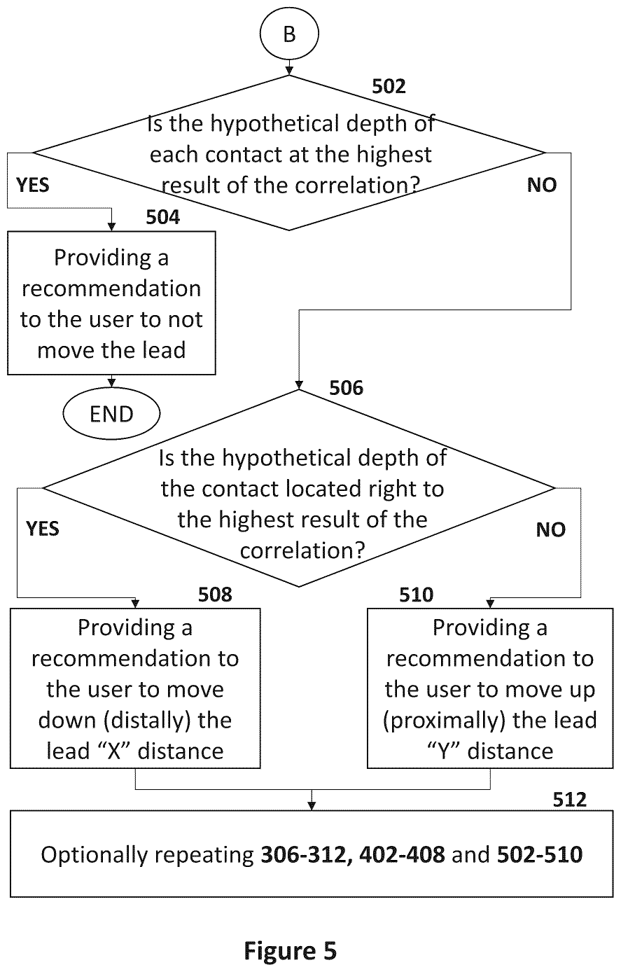

The flow chart continues following the letter “B” to FIG. 5.

In some embodiments, the assessment comprises assessing if the hypothetical depth of the contact matches the highest result of the correlation 502.

In some embodiments, if the answer is YES, then, the system provides a recommendation to the user to not move the lead anymore 504. And, in some embodiments, the method ends there.

In some embodiments, if the answer is NO, then, the assessment further comprises assessing if the hypothetical depth of the contact is located “right” to the highest correlation result, as seen in the correlation graph 506 (see explanation on correlation herein elsewhere).

In some embodiments, the threshold between providing a recommendation to move the lad or not is when a distance between a certain contact and the result of the highest correlation is of about 1 mm or more.

In some embodiments, if the answer is YES, then, the system provides a recommendation to the user to move down (distally) the lead a distance “X” according to the relative location of the lead to the highest correlation result, as seen in the correlation graph 508.

In some embodiments, if the answer is NO, then, the system provides a recommendation to the user to move up (proximally) the lead a distance “Y” according to the relative location of the lead to the highest correlation result, as seen in the correlation graph 510.

In some embodiments, optionally, steps 306-312, 402-408 and 502-510 are repeated at different depths 512, until the calculated hypothetical depth of the contact of the lead matches the highest correlation result and the system recommends not to move the lead anymore 504.

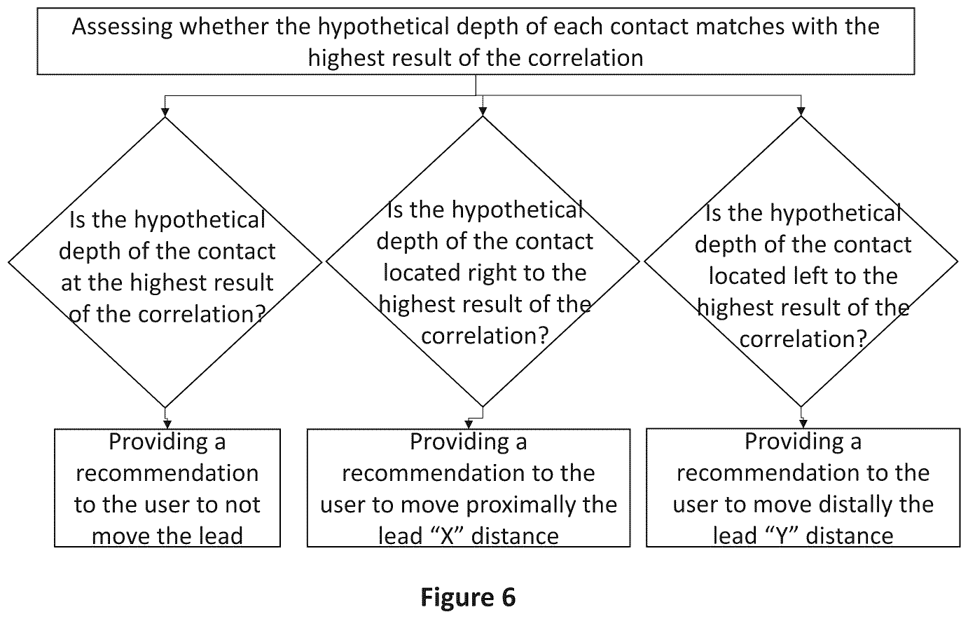

In some embodiments, the assessments of the location of the hypothetical depth, whether it matches or is on the right side in the graph or is on the left side in the graph, in relation of the highest correlation result, are performed in parallel, optionally independently to each other, and not one after the other as disclosed above, as schematically shown in FIG. 6.

### Exemplary Correlation

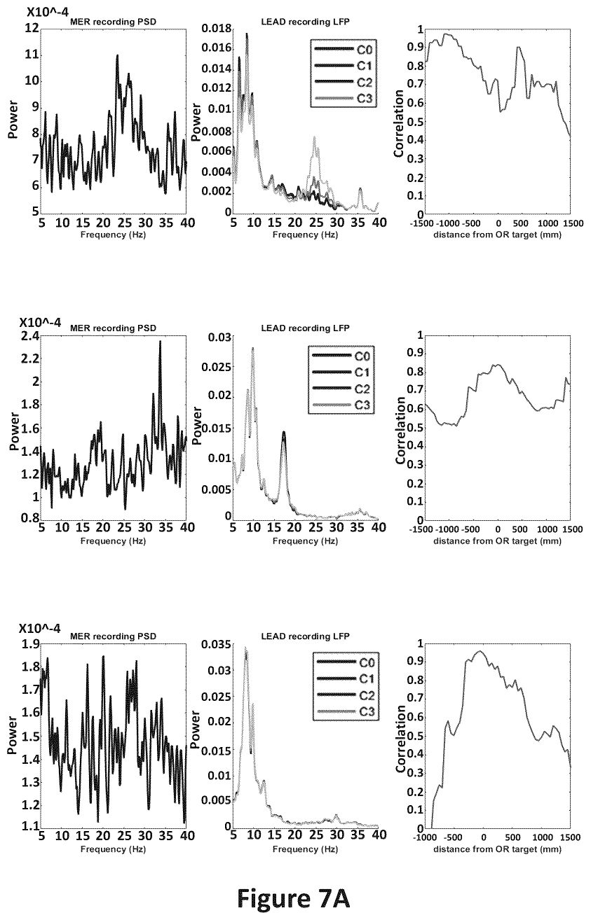

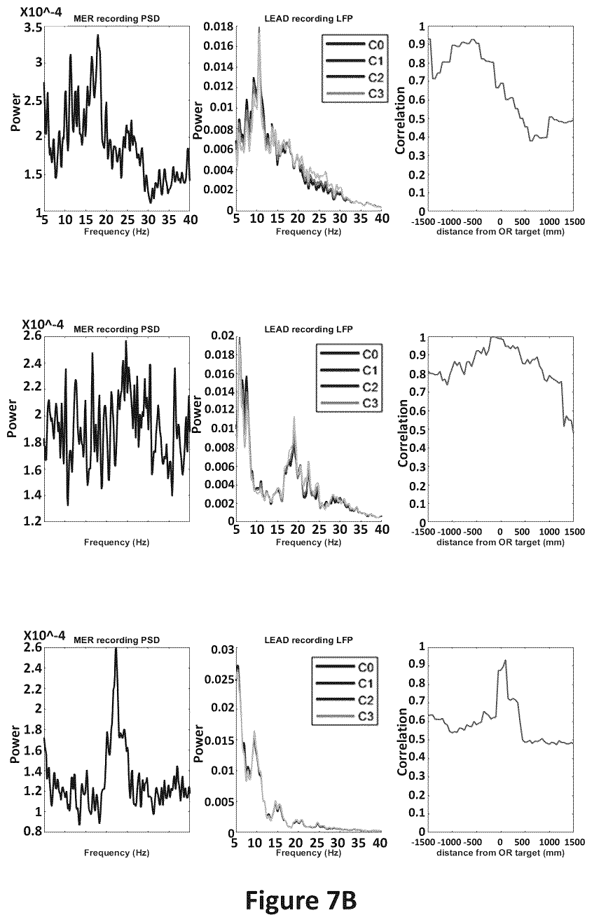

Referring now to FIGS. 7A-7B, showing exemplary graphs of MER recording PSD, lead recording LFP (Local Field Potential) and an exemplary correlation graph, according to some embodiments of the invention. In some embodiments, as mentioned above, the recordings made with the microelectrode are correlated with the recordings made with the lead in order to assess whether the current location of the lead is the optimal location. FIGS. 7A-7B show six different correlations. The graphs on the left show the MER recording PSD for a single frequency, while the graphs in the middle show the Lead recording LFP for the same frequency, and lastly, the graphs on the right show the correlation between the MER recording PSD and the Lead recording LFP for that frequency.

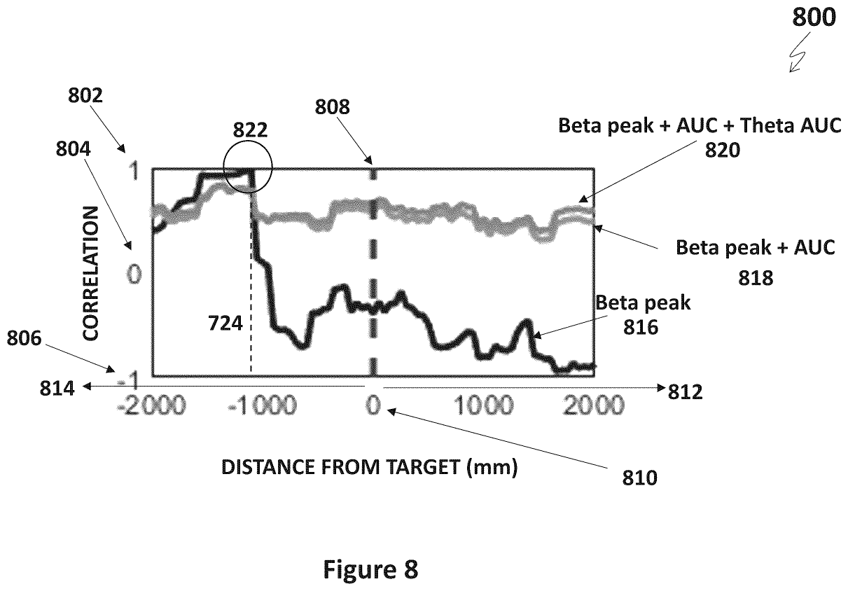

Referring now to FIG. 8, showing an exemplary graph, plotting the correlation between recordings made by the microelectrode and the lead in multiple frequencies, according to some embodiments of the invention. In some embodiments, the system is configured to plot the correlation in a graph 800, as shown for example in FIG. 8. The X axis in the represents the distance of the lead from the optimal target, measured for example in millimeters (mm), while the Y axis represents the level of correlation between the recordings previously made by the microelectrode and the recordings made by the lead in real-time, where “1” (802) represents the maximum correlation, “0” (804) represents the minimum correlation, and below “0” until “−1” (806) means that no correlation was able to be made. The graph 800 shows a center dotted line 808 meeting the “0” (810) in the X axis, which represents the current location of the lead. Moving to the positive numbers (arrow 812) in the X axis means moving the lead distally (meaning more deep), while moving to the negative numbers (arrow 814) in the X axis means moving the lead proximally (meaning less deep-extracting the lead). The exemplary graph 800 in FIG. 8 shows 3 plotted lines, 1. Representing the Correlations in Beta peak (816), 2. Representing the Correlations in Beta peak+Beta AUC (818), and 3. Representing Beta peak+Beta AUC+Theta AUC (820). Focusing only on the plotted line for the Beta peak 818, it can be seen that the maximum correlation, pointed out by circle 822, is located about −1100 mm from the location of the lead, as schematically shown by dotted line 824. In this example, the system will communicate the user that in order to achieve maximum correlation, the lead needs to the extracted/moved proximally about 1100 mm from its current location.

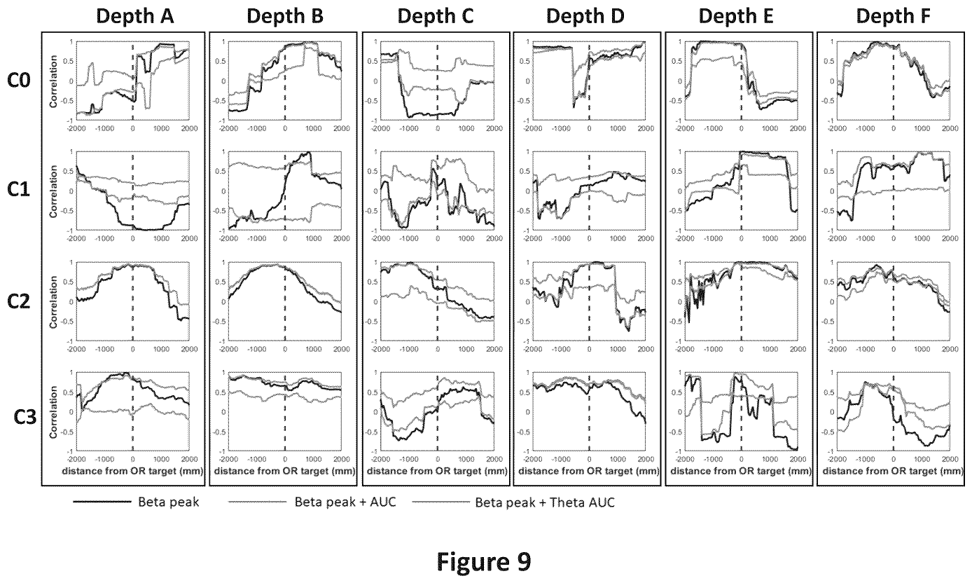

Referring now to FIG. 9, showing a plurality of exemplary calculated correlations for a plurality of frequencies as generated by the system, according to some embodiments of the invention. FIG. 9 shows exemplary calculations of correlation made for 4 distinct contacts (C0, C2, C2, C3) made at different depths (A, B, C, D, E, F), each correlation made for 3 distinct frequencies (Beta peak, Beta peak+Beta AUC, Beta peak+Theta AUC). In some embodiments, the system is configured to provide a recommendation for each contact at each depth for each of the frequencies. In some embodiments, the user utilizes the correlations to choose which contact will be used for the stimulation according to the requirements. In some embodiments, the user chooses the location according to graph and according to the patient's symptoms that are needed to be treated.

In some embodiments, a plurality of graphs are generated in real-time corresponding to each of the contacts in the lead. In some embodiments, the generated graphs are not shown in the graphical interface unit (GUI) of the user, rather only the recommendations (either move the lead forwards or backwards and how much). In some embodiments, the user may choose to look at the graphs. In some embodiments, the graphs are shown to the user.

In some embodiments, the estimated location of the lead is also assessed by comparing the results of the consecutive recordings made by the microelectrode with the results of the consecutive recordings made by the lead, optionally by using the results of the correlation. For example, just to simplify the explanations: if the recordings by the microelectrode would be translated to letters, the consecutive recordings would show for example something like this: BBTBBTTTB. Then, when inserting the lead, the lead would begin recording and showing first the B, then another B, then a T, then a B, and so on. Since each of the recording made by the microelectrode is performed at a known depth, the system can calculate the current depth of the lead according to the sequence of recordings being recorded in real-time by the lead and comparing them with the recording previously made with the microelectrode. In some embodiments, the system is configured to utilize the historical sequence of recordings made by the microelectrode to later assess the location of the lead according to the sequence of recordings being recorded.

In some embodiments, the correlation can be made using one or more frequencies. For example, utilizing one or more of Beta (13 Hz to 35 Hz) peak power, Beta area under the curve (AUC), Theta (4 Hz to 8 Hz) peak power, Theta area under the curve (AUC), Gamma (35 Hz to 60 Hz) peak power and Gamma area under the curve (AUC). In some embodiments, the reason to use or another depends on one or more of: the area that needs stimulating, the type of disease being treated, how easy is to read the recordings, the levels of interference in the target.

For example, for patients suffering of subthalamic nucleus Parkinson's disease, and/or Parkinsonian rigidity and bradykinesia, stimulations are performed in the Beta frequency in order to treat the symptoms. Therefore, correlations will be made especially for the Beta frequencies. Another example, for patients suffering from tremors, stimulations are performed in the Theta frequency in order to treat the symptoms. Therefore, correlations will be made especially for the Theta frequencies.

### Exemplary Gui for Users and Methods for Performing the Procedure

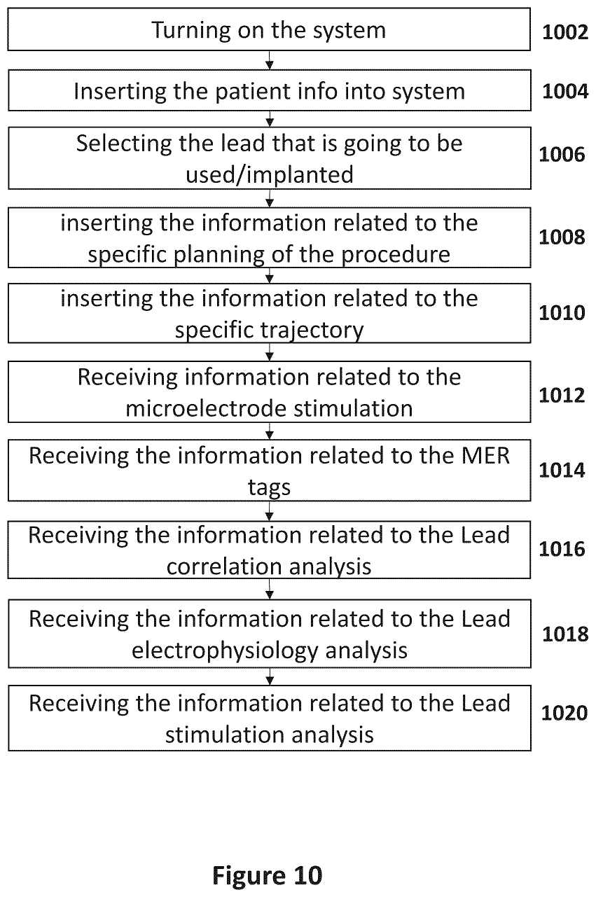

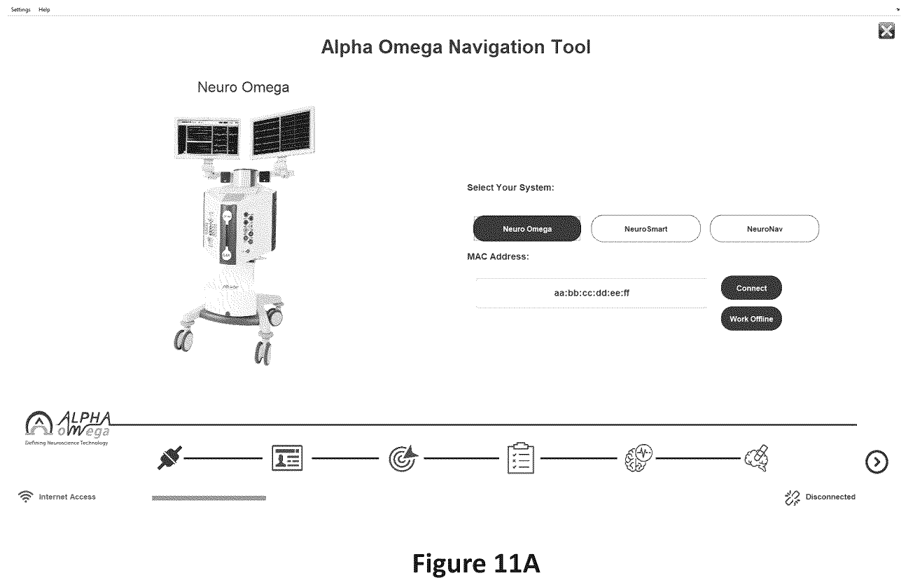

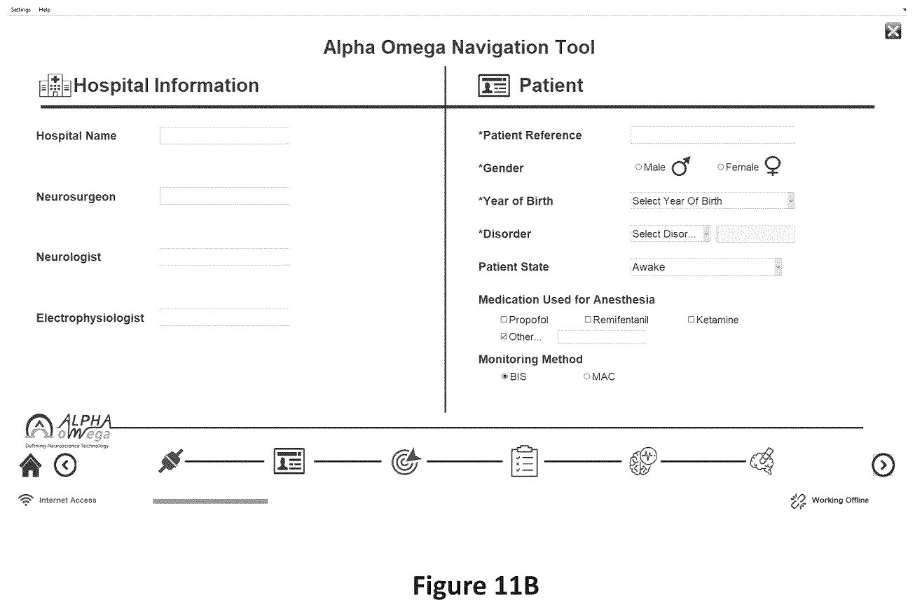

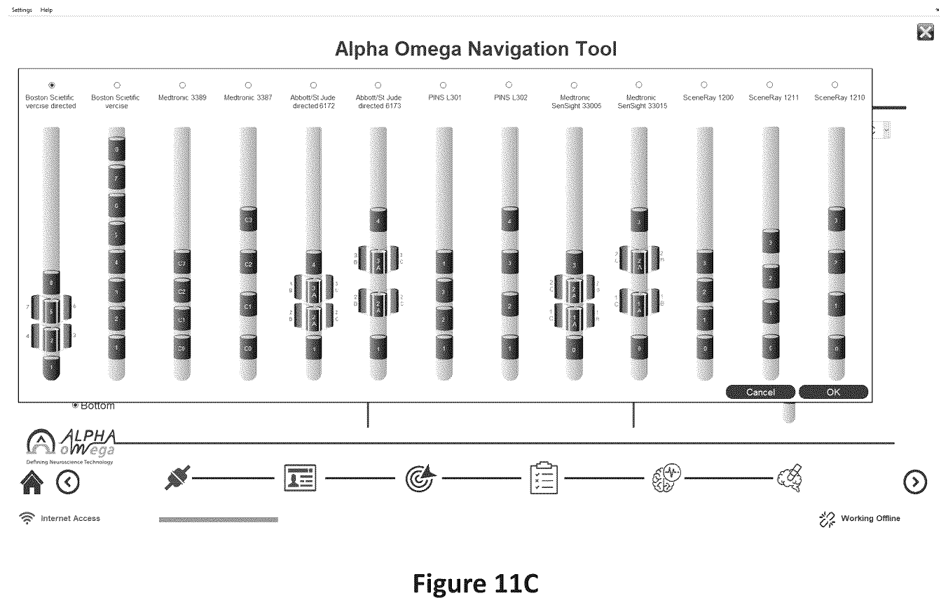

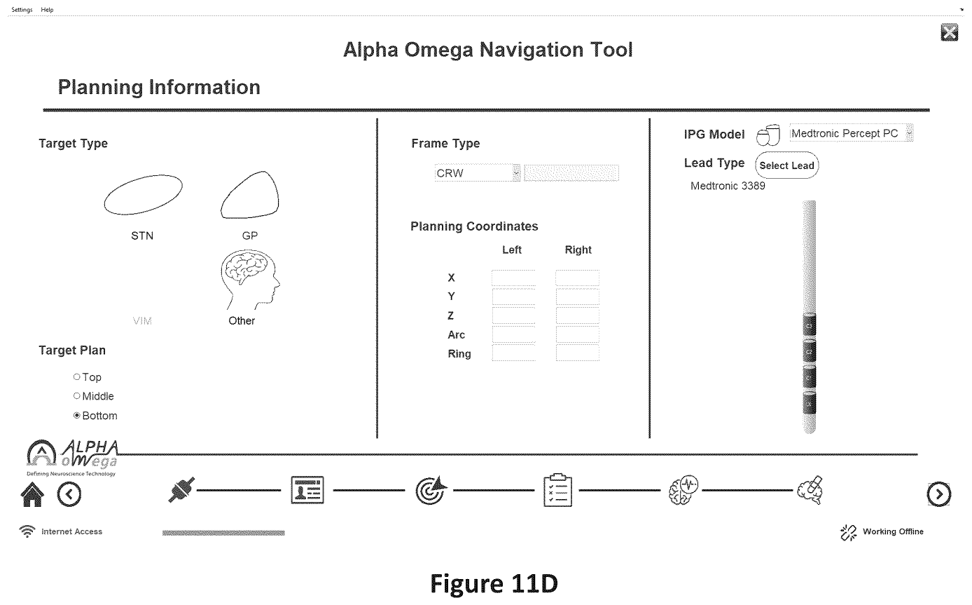

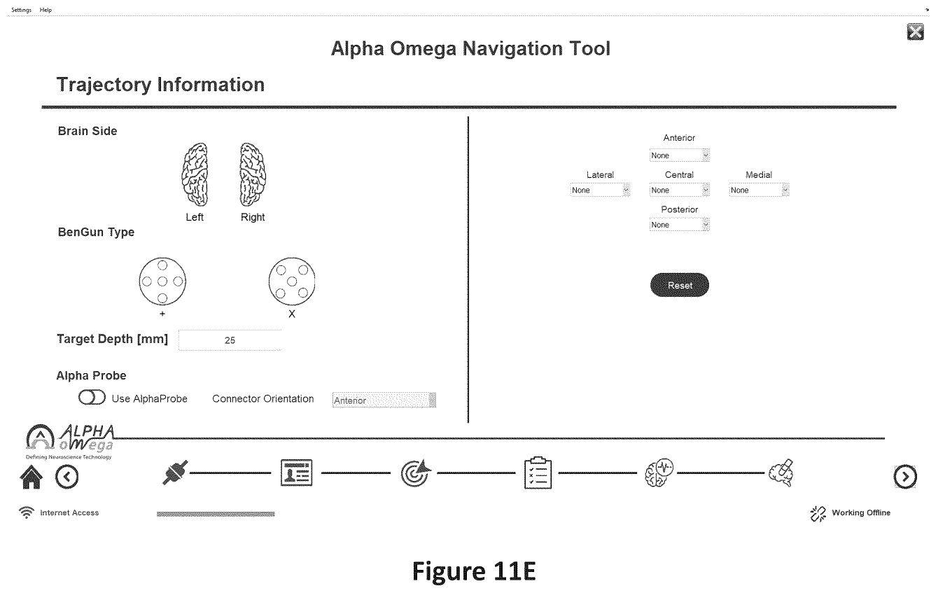

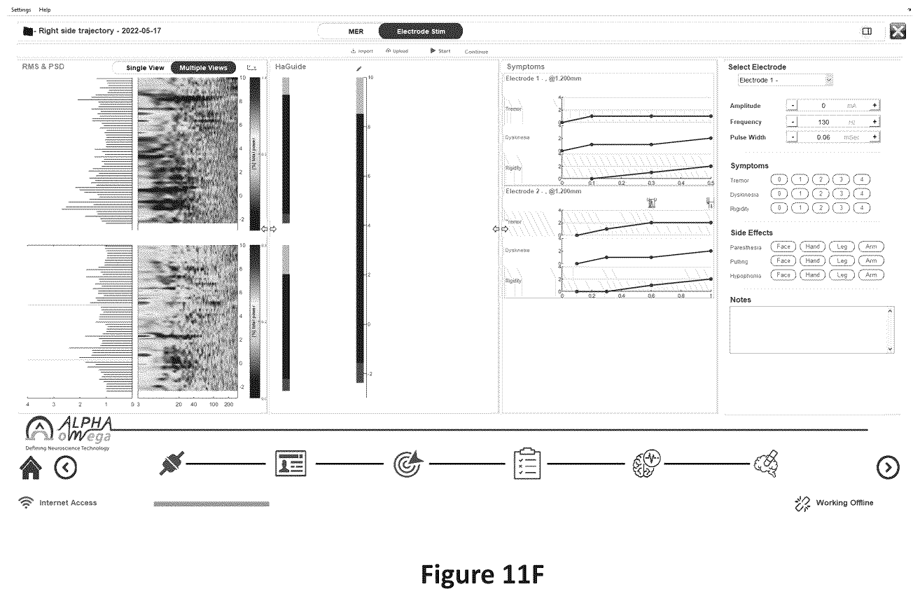

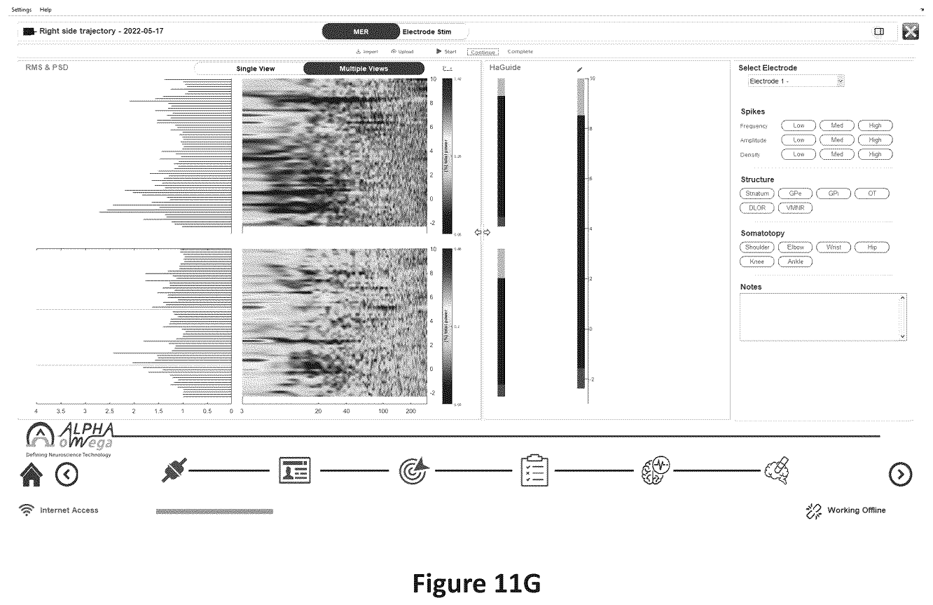

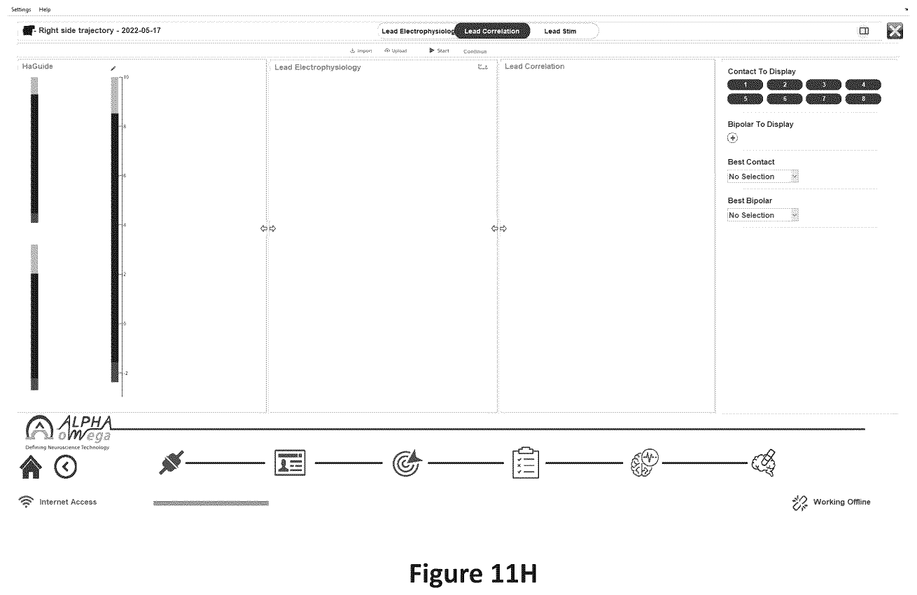

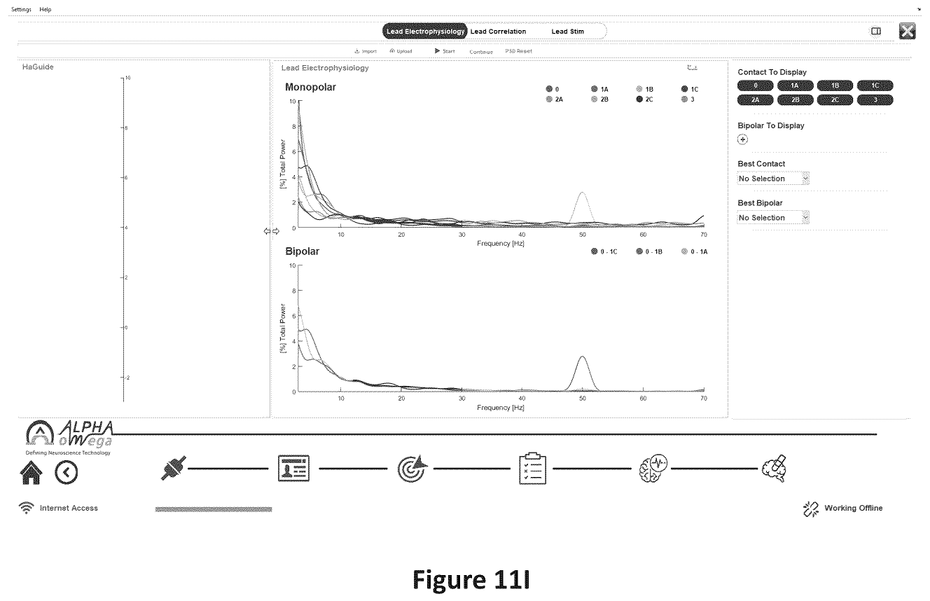

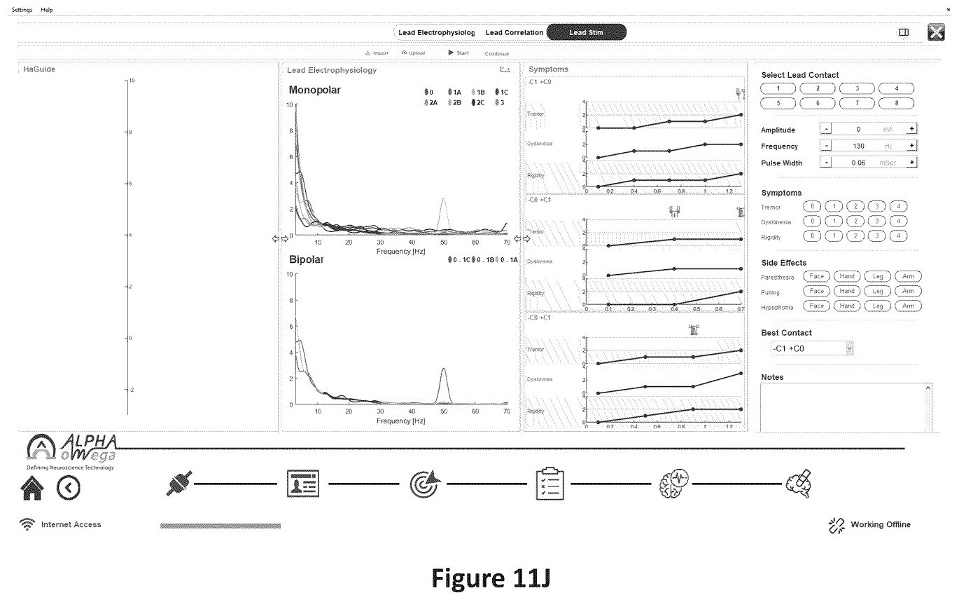

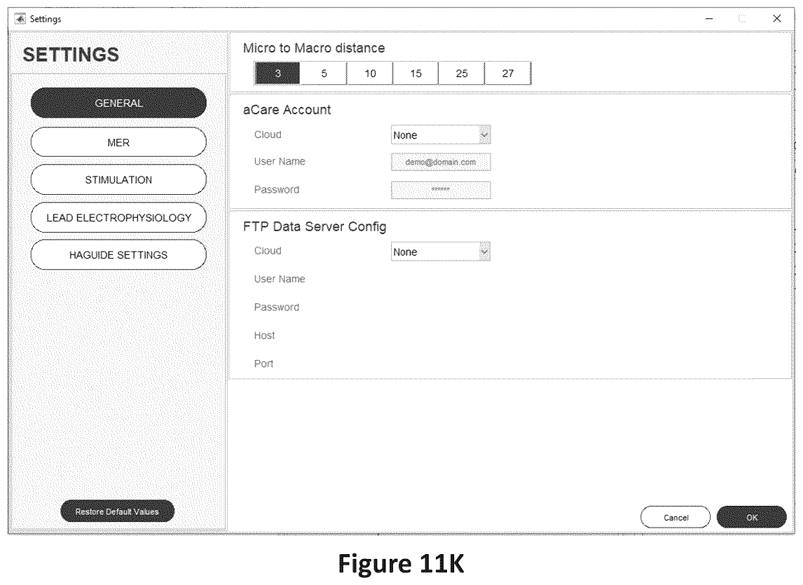

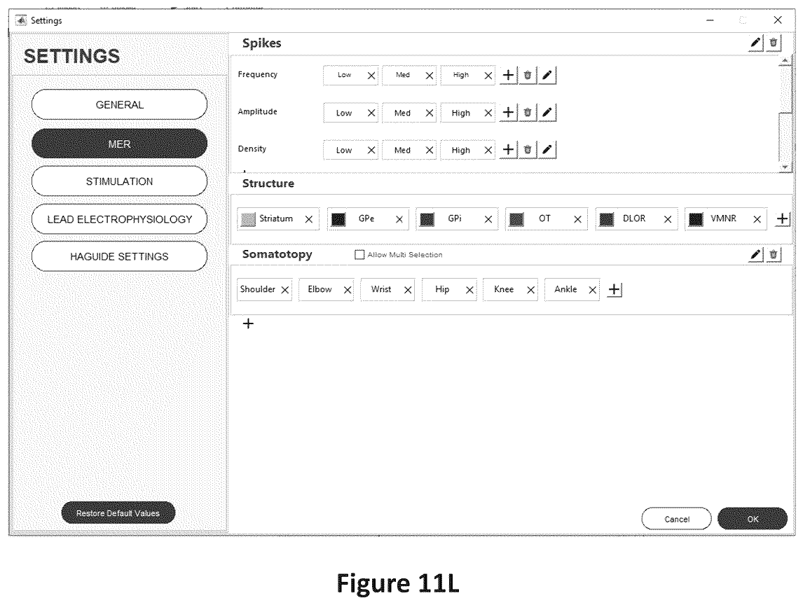

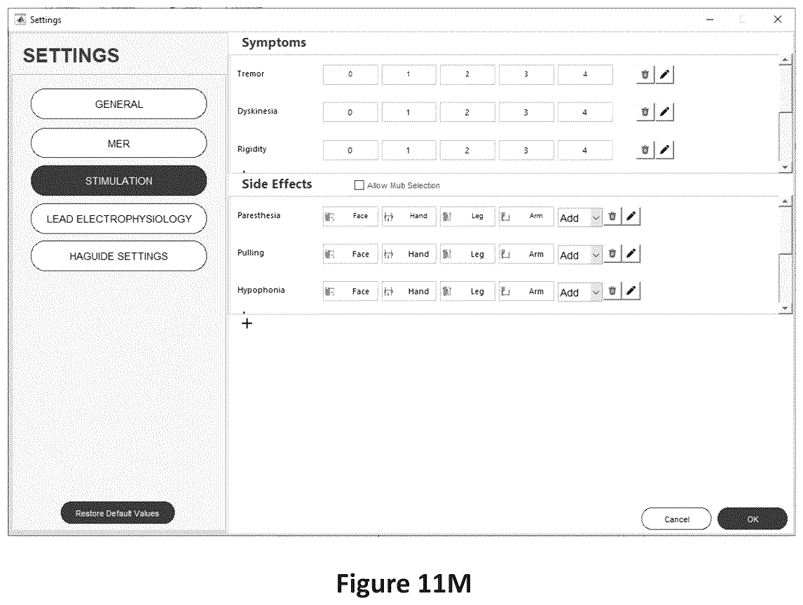

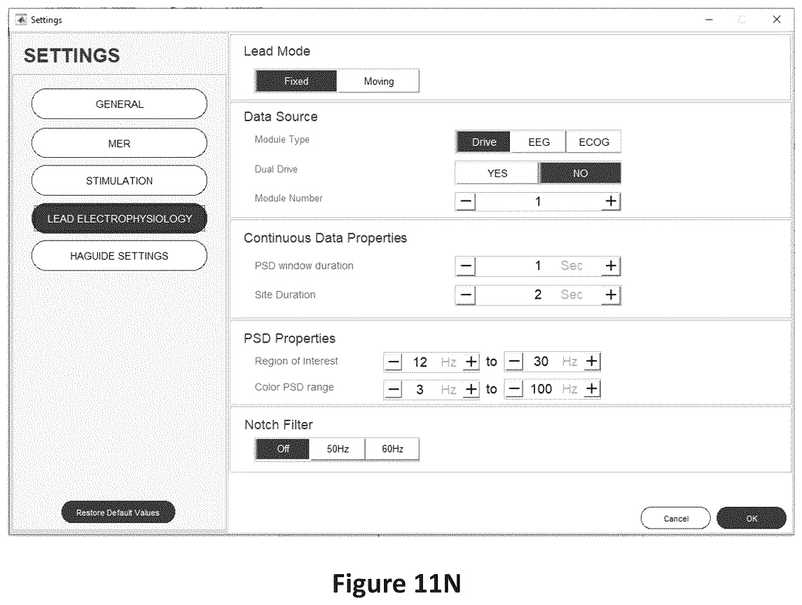

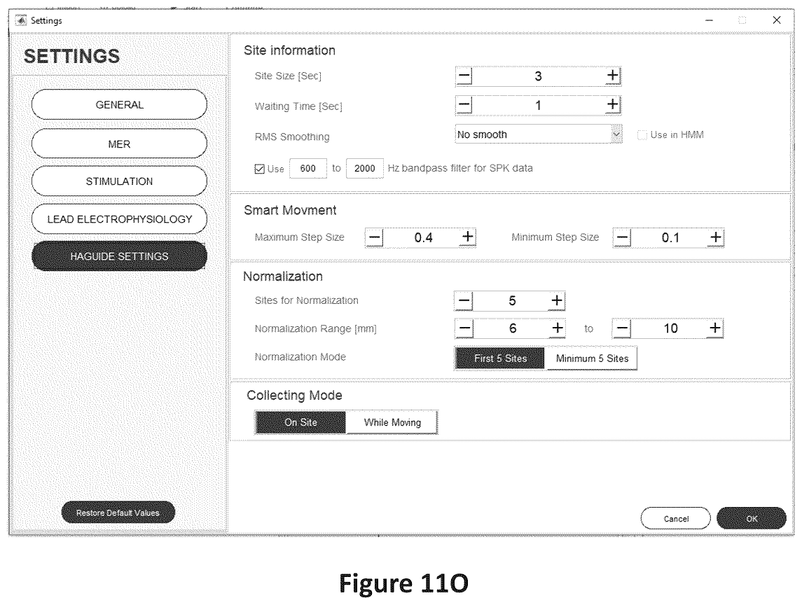

Referring now to FIG. 10 showing a flowchart of an exemplary procedure, according to some embodiments of the invention; additionally, referring to FIGS. 11A-110, showing exemplary graphic user interface screenshots related to the exemplary procedure, according to some embodiments of the invention.

In some embodiments, the user turns on the system 1002. FIG. 11A shows an exemplary screenshot of the image shown to the user when turning on the system.

In some embodiments, the user inserts the information related to the specific patient 1004 (if not already in the system). In some embodiments, if the information related to the patient was already inserted in the system, the user can search and open the dedicated file related to the specific patient. FIG. 11B shows an exemplary screenshot of the image shown to the user to insert the information related to the specific patient into the system.

In some embodiments, the user selects the lead that is going to be used/implanted 1006, for example from a pre-set number of leads. FIG. 11C shows an exemplary screenshot of the image, having a plurality of types of leads, shown to the user when providing the choice of which lead is going to be used.

In some embodiments, the user inserts the information related to the specific planning of the procedure 1008 (if not already in the system). In some embodiments, if the information related to the planning of the procedure was already inserted in the system, the user can search and open the dedicated file related to the specific planning of the procedure. In some embodiments, information related to the planning of the procedure comprises, for example, one or more of: the target type (for example the Subthalamic Nucleus (STN), the Globus pallidus (GP), or other); the frame type; the lead model, etc. FIG. 11D shows an exemplary screenshot of the image shown to the user to insert the information related to the specific patient into the system.

In some embodiments, the user inserts the information related to the specific trajectory the user will or should take during the procedure 1010 (if not already in the system). In some embodiments, if the information related to the specific trajectory was already inserted in the system, the user can search and open the dedicated file related to the specific trajectory. In some embodiments, information related to the specific trajectory comprises, for example, one or more of: the brain side; the BenGun type; the, optionally estimated, target depth (for example, measured in millimeters); coordinates for the location if insertion; etc. FIG. 11E shows an exemplary screenshot of the image shown to the user to insert the information related to the specific trajectory into the system.

In some embodiments, the user receives the information related to the microelectrode stimulation 1012. FIG. 11F shows an exemplary screenshot of the image shown to the user having the information regarding the microelectrode stimulation.

In some embodiments, the user receives the information related to the MER tags 1014. FIG. 11G shows an exemplary screenshot of the image shown to the user having the information regarding MER tags. In some embodiments, a potential advantage of providing these tags is that they potentially help the user to write his comments in a quick way. In some embodiments, the user can press on predefined tags like “STN” or “Rigidity” and the tag will appear in the relevant depth the user clicked.

In some embodiments, the user receives the information related to the Lead correlation analysis 1016. FIG. 11H shows an exemplary screenshot of the image shown to the user having the information regarding the Lead correlation analysis.

In some embodiments, the user receives the information related to the Lead electrophysiology analysis 1018. FIG. 11I shows an exemplary screenshot of the image shown to the user having the information regarding the Lead electrophysiology analysis. In some embodiments, in this screen, the recordings from the lead are displayed, for example monopolar view or bipolar view.

In some embodiments, the user receives the information related to the Lead stimulation analysis 1020. FIG. 11J shows an exemplary screenshot of the image shown to the user having the information regarding the Lead stimulation analysis. In some embodiments, in this screen, recordings from the lead are shown and the user is allowed to write the symptom assessment after the stimulation. In some embodiments, the stimulation is performed from the other software (no navigation tools) and the user may just save the symptom assessment.

FIGS. 11K, 11L, 11M, 11N and 11O, show exemplary screenshots of the exemplary plurality of settings that the user is allowed to set for the procedure, for example, user account settings, the MER information settings, the stimulation information settings, the lead electrophysiology settings and the settings of the algorithm.

As used herein with reference to quantity or value, the term “about” means “within ±20 % of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-27. (canceled)

28. A system for implanting a lead in a brain of a patient, comprising:
a. at least one microelectrode;
b. at least one lead comprising at least one contact
c. circuitry comprising instructions for:
i. receiving at least one first reading using at least one microelectrode at a first location;
ii. receiving at least one chosen target location for at least one lead comprising at least one contact according to said at least one first reading;
iii. receiving at least one second reading at a second location at or near to said received chosen target location using said at least one contact of said at least one lead;
iv. performing a correlation between said at least one first reading and said at least one second reading;
v. estimating a distance between said first location and said second location using a result of said correlation;
vi. providing information regarding one or both of a distance and a direction between a location of said at least one contact of said at least one lead to said chosen target location.

29. The system according to claim 28, wherein said providing information comprises providing information regarding a direction to move said at least one contact of said at least one lead to reach said chosen target location.

30. The system according to claim 28, wherein said providing information comprises providing information regarding a distance to move said at least one contact of said at least one lead to reach said chosen target location.

31. The system according to claim 28, wherein said first location is said target location.

32. The system according to claim 28, wherein when said at least one lead comprises more than one contact, and said steps c-f are performed for each contact.

33. The system according to claim 28, wherein before said performing at least one first reading, the method further comprises inserting said at least one microelectrode to an estimated location within said brain of said patient.

34. The system according to claim 28, wherein before said performing at least one second reading, the method further comprises extracting said at least one microelectrode and inserting said at least one lead comprising said at least one contact using a same path as said at least one microelectrode until reaching a location close to said chosen target location.

35. The system according to claim 28, wherein said at least one first reading is performed in at least one frequency; and wherein said at least one frequency is one or more of Beta, Theta and Gamma.

36. (canceled)

37. The system according to claim 35, wherein said at least one second reading is performed in a same frequency as said at least one first reading.

38. The system according to claim 28, wherein said performing at least one first reading further comprises obtaining a plurality of microelectrode Power Spectral Densities (PSDs) of said at least one first reading.

39. The system according to claim 28, wherein said performing at least one second reading further comprises obtaining at least one lead PSDs of said of said at least one second reading.

40. The system according to claim 38, wherein said correlating comprises calculating a group of microelectrode PSDs from said plurality of microelectrode PSDs that match said at least one lead PSDs.

41. The system according to claim 40, wherein said correlating comprises, after said calculating a group of microelectrode PSDs, averaging values of said calculated group of said microelectrode PSDs.

42. The system according to claim 41, wherein said correlating comprises correlating between said averaged PSDs values and said at least one lead PSDs;
wherein said correlating comprises one or more of:
a. calculating a hypothetical depth in which said at least one contact of said at least one lead is located based on a result of said correlation;
b. calculating a distance between said at least one contact of said at least one lead to said chosen location based on a result of said correlation;
wherein said distance is calculated according to a distance between a location of said at least one contact and a location where a result of said correlation is a highest result of said correlation.

43-45. (canceled)

46. The system according to claim 28, wherein said performing at least one first reading comprises performing a plurality of readings at fixed times using at least one microelectrode; and wherein said a plurality of readings are performed every 0.1 seconds.

47. (canceled)

48. The system according to claim 28, wherein said performing at least one first reading comprises performing a plurality of readings at fixed depths using at least one microelectrode; and wherein said plurality of readings are performed every 0.1 millimeters.

49. (canceled)

50. The system according to claim 38, wherein said plurality of microelectrode Power Spectral Densities (PSDs) are obtained for a plurality of depths to which said at least one microelectrode is inserted.

51. The system according to claim 38, wherein said plurality of microelectrode Power Spectral Densities (PSDs) are obtained using a pWelch method.

52. The system according to claim 28, wherein said correlation is performed using one or more of Beta peak power, Beta AUC, Theta peak power, Theta AUC, Gamma peak power and Gamma AUC.

53. The system according to claim 28, wherein said performing a correlation is performed multiple times at different depths.
